(19) United States
(12) Patent Application Publication
(10) Pub. No.: US 2019/0175904 A1
(43) Pub. Date: Jun. 13, 2019

(54) SPINAL CORD STIMULATION

(71) Applicant: BIOTRONIK SE &. Co. KG, Berlin (DE)

(72) Inventors: Marcelo BARU, Tualatin, OR (US); Alan FRYER, Portland, OR (US)

(73) Assignee: BIOTRONIK SE &. Co. KG, Berlin (DE)

(21) Appl. No.: 16/218,802

(22) Filed: Dec. 13, 2018

Related U.S. Application Data
(60) Provisional application No. 62/597,947, filed on Dec. 13, 2017.

(51) Int. Cl. A61N 1/05; A61N 1/36

(52) U.S. Cl. CPC A61N 1/0551; A61N 1/36071; A61N 1/36171; A61N 1/37211; A61N 1/36157; A61N 1/36139; A61N 1/36178

(57) ABSTRACT

An implantable pulse generator configured to deliver first and second stimulation pulse trains wherein the first and second trains are delivered simultaneously, are delivered at different frequencies, and are each biphasic, including a stimulation phase followed by a balancing phase.

This nonprovisional application claims priority to U.S. Provisional Application No. 62/597,947, which was filed on Dec. 13, 2017, and which is herein incorporated by reference.

### BACKGROUND OF THE INVENTION

### Field of the Invention

The present invention relates to an invention relating generally to spinal cord stimulation (SCS) systems, and more specifically to multi-electrode SCS systems capable of simultaneous delivery of different stimuli (different stimulation frequencies, waveforms, etc.) from different electrodes.

### Description of the Background Art

Conventional spinal cord stimulation (SCS) systems use electrodes to supply electrical pulses to the dorsal column fibers within the spinal cord, typically for the purpose of stimulating sensory nerves associated with a painful area of the body. The stimulation can induce paresthesia over the painful area, masking the pain and replacing it with a tingling sensation. Early SCS systems typically delivered voltage-based, tonic frequency stimulation using bipolar electrodes (i.e., a stimulation electrode and a return electrode), an arrangement adopted from the pacemaker industry. Current SCS systems often have multiple electrodes that can simultaneously deliver current-based stimulation using multiple sourcing and sinking currents of different amplitudes, allowing cathode (sinking) and anode (sourcing) electrodes to have different weights to steer the electrical field of a stimulation pattern to different regions as desired. Current steering beneficially permits adjustment of paresthesia coverage to overlap the painful area as much as possible. See, e.g., U.S. Pat. Nos. 6,909,917 and 6,516,227. Other developments include high frequency SCS (in the tens of kHz range), which has been claimed to calm pain without paresthesia (e.g., U.S. Pat. No. 8,209,021), and SCS using interferential currents (IFCs) (e.g., U.S. Pat. No. 8,977,363). IFCs utilize two independent alternating (e.g., sinusoidal) currents with frequencies in the range of 500 Hz to 20,000 Hz that are injected diagonally of each other, creating an X pattern. When these are combined in tissue, they result in a beat frequency of up to 250 Hz with deeper tissue penetration, and theoretically having larger amplitude where the two currents intersect.

U.S. provisional application 62/476,884 by the same inventors inter alia as the present application (the entire contents of which are incorporated by reference into this document), relates to a device including a novel parameter for titration of neuro stimulation, wherein the parameter is based on paresthesia sensation of the patient.

U.S. provisional application 62/476,885 by the same inventors inter alia as the present application (the entire contents of which are incorporated by reference into this document), relates to a multiple electrode stimulation scheme for neuromodulation having reduced energy consumption and effective charge balanced stimulation.

SCS systems may simultaneously stimulate different nerves which affect different body areas, e.g., the lower back and legs. It may be useful to apply different stimulation to the different nerves, in particular, different trains of electrical pulses having different frequencies, amplitudes, and/or waveform shapes. Some or all of the different trains may share electrodes. If the trains have the same frequency, the pulses of the different trains can typically be delivered without interfering with each other, as pulses of one train can simply follow pulses of another train. See, e.g., U.S. Pat. No. 8,209,021, wherein multiple trains having the same frequency are delivered multiplexed in time. However, where trains involve different frequencies, train “arbitration” methods are needed to deliver the trains simultaneously without different trains' pulses overlapping. These arbitration methods tend to carry significant drawbacks. For example, in U.S. Pat. No. 6,516,227, a proposed arbitration method for trains having different frequencies temporarily interrupts the balancing phases of an active train if stimulation in another train is scheduled to be delivered. Balancing is the process by which charge injected into tissue by a stimulation pulse is subsequently reversed in tissue (withdrawn) to stop electrochemical reactions caused by the stimulation pulse and avoid tissue and/or electrode damage, and since charge withdrawal is preferably effected as soon as possible after the injected charge is effective (i.e., once it evokes an action potential), interruption of balancing is not preferred. Skipping pulses to avoid interference is also undesirable, as the effective stimulation frequencies may vary outside a comfort zone for the patient.

Another problem encountered in SCS systems is that of residual charge and “potential runaway” at the electrodes, particularly at high pulsing frequencies. As noted above, stimulation is typically performed by injecting a cathodic pulse, followed by anodic withdrawal of the injected charge. At low pulsing frequencies, this charge balancing can be passive: the electrodes are short-circuited and the injected charge can simply be drained off. Since this process can take time, passive balancing typically cannot be achieved at high pulsing frequencies because there is insufficient time between pulses to achieve passive balancing. Instead, at high frequencies, active balancing must be used, wherein the electrode's charge injection is reversed to actively withdraw the injected charge from tissue. The problem with active balancing is that it tends to be imperfect: some of the injected charge tends to be irreversibly lost. As a result, withdrawing charge equal to the injected charge tends to eventually result in increasing potentials across the electrode-tissue interface. This in turn increases irreversible Faradaic reactions at the electrode, which can give rise to tissue damage and electrode corrosion. This electrode potential runaway issue may be further enhanced by mismatches in the current drivers that implement charge injection (pulsing) and charge withdrawal (balancing); the pulsing and balancing may be mismatched by up to a few percent. These mismatches accumulate voltages in the DC blocking capacitors in series with the electrodes, a problem known as “voltage buildup,” which reduces the effective value of the capacitors. Voltage buildup may also force the electronics driving them above the maximum voltage used in the SCS system, or below system ground, which will activate protection diodes in the driving electronics if not handled properly.

### SUMMARY OF THE INVENTION

The invention is directed to spinal cord stimulation (SCS) systems which can address one or more of the aforementioned problems. An exemplary preferred version of the invention involves an SCS system including an implantable pulse generator (IPG) with a hermetically sealed case preferably having at least one electrical conductive area, and also having a header accepting the connection of implantable leads. At least one implantable lead connected to the header has multiple electrodes located at its distal end, whereby electrical stimulus generated by the IPG can be delivered through the electrodes. The IPG preferably delivers simultaneous multi-electrode current-based stimulation, which can be delivered at high pulsing rates (e.g., kHz range), and under closed-loop control wherein the electrode potentials are maintained within a safe voltage operating window with avoidance of voltage buildup in the output coupling capacitors (thereby allowing for uninterrupted therapy delivery). Different stimulation can be provided to different nerve areas, where each nerve area represents a target region in the body. Stimulation may be performed using conventional (e.g., rectangular) pulse trains, or via custom waveforms chosen by the IPG programmer. A train arbitration method allows simultaneous stimulation of multiple nerve areas at independent frequencies without interruption of phases in trains, i.e., trains run transparently of each other.

The IPG is preferably capable of switching between different stimulation programs with different stimulation parameters based on the output of a posture sensor on the IPG module. This allows for automatic therapy adjustment according to the patient's body position to account for variation in stimulation intensity as body position changes. A patient remote control may also be used to wirelessly switch between the different stimulation programs. The IPG preferably has transcutaneous communication and battery recharging capabilities, whereby external devices may communicate data and/or power wirelessly to and/or from the IPG.

The IPG is also preferably able to deliver adaptive therapy based on feedback from neural responses to stimulation, more specifically from evoked compound action potentials (ECAPs), whereby therapy can be adjusted without the need for patient interaction. High-resolution ECAP recording permits assessment of electrode lead migration, automatic adjustments to therapy in response to detected lead migration, and optimum sub-perception threshold therapy programming by recording ECAP while delivering kHz stimulation. In particular, therapy intensity may be varied to accommodate lead (electrode) movement arising from respiration relative to target nerve fibers.

The IPG therefore closes the gap between low and high frequency implantable SCS systems by delivering simultaneous multi-electrode current-based stimulation across the entire frequency range of present SCS systems. The use of closed-loop stimulation control allows such delivery without therapy interruption while guaranteeing safe electrode and tissue operation. The IPG can utilize a typical IPG front end incorporating direct current (DC) blocking capacitors, thereby better ensuring safety.

The IPG may also implement a train arbitration method that does not require interruption of a train balancing phase if another train is scheduled to deliver a stimulation phase. This permits simultaneous independent asynchronous trains which target different body areas, and which may share electrodes. The arbitration method for two trains A, B uses two sets of biphasic pulses A(n) and B(n), which can occur in any order. During the first biphasic pulse of a set (n), the balancing phase of the second biphasic pulse of set (n) is determined. During the second biphasic pulse of a set (n), the balancing phase of the first biphasic pulse of the set (n+1) is determined instead. Where one train has a higher frequency than the other, for example where train B has a higher frequency than train A, if B(n) is a first biphasic pulse of a set (n) and delivery of B(n+1) is scheduled before A(n), biphasic pulse B(n+1) is delivered and assumes the role of B(n), redefining the set (n) to resume the arbitration method. Arbitration of additional pairs of trains occurs in a similar fashion. In all cases, periods are adjusted (frequency is jittered) to allow delivery of passive balancing when possible, and active balancing otherwise, thereby minimizing power consumption. Balancing is not interrupted to deliver an upcoming biphasic pulse.

Therefore, according to a first aspect of the invention, an implantable pulse generator (IPG) is disclosed which is configured to deliver first and second stimulation pulse trains, wherein the first and second trains A, B

(1) are delivered simultaneously,
(2) are delivered at different frequencies,
(3) are each biphasic, including a stimulation phase followed by a balancing phase.

Preferably, the IPG according to the invention is configured such that following each train's stimulation phase:

(1) the train's balancing phase is passively delivered if passive delivery can be completed prior to the other train's stimulation phase;
(2) the train's balancing phase is actively delivered.

According to an embodiment of the invention, the train's balancing phase is actively delivered with rescheduling of the other train's stimulation phase if:

i: passive delivery cannot be completed prior to the other train's stimulation phase, and

ii. active delivery cannot be completed prior to the other train's stimulation phase.

According to a preferred embodiment of the invention, the train's balancing phase is actively delivered without rescheduling the other train's stimulation phase if:

i: passive delivery cannot be completed prior to the other train's stimulation phase, and

ii. active delivery can be completed prior to the other train's stimulation phase.

According to an embodiment of the invention, the implantable pulse generator (IPG) further includes an electrode through which both the first and second trains A, B are delivered. Preferably, a lead comprises the electrode, wherein the lead is electrically connected to the IPG.

According to an embodiment of the invention, the IPG is configured such that rescheduling of any biphasic stimulation pulse changes (jitters) the trains' stimulation frequencies f_{Stim(s)} less than ±20%.

Moreover, according to an aspect of the invention, a method for generating electrical pulses for neuro stimulation is disclosed, comprising delivery of first and second stimulation pulse trains wherein the first and second trains A, B

(1) are delivered simultaneously,
(2) are delivered at different frequencies,
(3) are each biphasic, including a stimulation phase followed by a balancing phase.

Preferably, the method for generating electrical pulses according to the invention further comprises that following each train's stimulation phase:

(1) the train's balancing phase is passively delivered if passive delivery can be completed prior to the other train's stimulation phase;
(2) else the train's balancing phase is actively delivered.

According to an embodiment of the invention, the inventive method further comprises that the train's balancing phase is actively delivered with rescheduling of the other train's stimulation phase if:

i: passive delivery cannot be completed prior to the other train's stimulation phase, and

ii. active delivery cannot be completed prior to the other train's stimulation phase.

According to a preferred embodiment of the invention, the inventive method further comprises that the train's balancing phase is actively delivered without rescheduling the other train's stimulation phase if:

i: passive delivery cannot be completed prior to the other train's stimulation phase, and

ii. active delivery can be completed prior to the other train's stimulation phase.

According to an embodiment of the invention, the inventive method further includes that both the first and second trains A, B are delivered via an electrode.

According to an embodiment of the invention, the inventive method comprises that rescheduling of any biphasic stimulation pulse changes (jitters) the trains' stimulation frequencies f_{Stim(s)} less than ±20%.

According to an aspect of the present invention, a system is disclosed, wherein the device system includes:

an implantable medical device, wherein the implantable medical device comprises an implantable pulse generator (IPG) according to at least one of the claims 1 to 6,

at least one lead wherein at least one electrode for electrical stimulation is located along the elongated lead body and/or the distal end, and wherein the lead proximal end is electrically connectable to the implantable medical device,

wherein the IPG is electrically connected to the electrode such that stimulation pulse trains can be delivered via the at least one electrode.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 schematically illustrates an exemplary implantable spinal cord stimulation (SCS) system wherein the invention may be implemented, illustrating the system's implantable pulse generator (IPG) 104 and its leads 101 as they might be situated when implanted within a human body, along with a clinician programmer 106.a, a patient remote 106.b, and a charger 110 usable to communicate power and/or data to and/or from the IPG 104.

FIG. 2 provides a schematic block diagram of possible architecture of the IPG 104 of FIG. 1.

FIG. 3a illustrates exemplary rectangular stimulation phases 300 that might be delivered by the IPG 104 of FIG. 1, along with a passive balancing phase 301; and

FIG. 3b illustrates the same, but using an active balancing phase 302.

FIGS. 4, 4A, 4B and 4C illustrate the exemplary delivery timing of a pair of stimulation pulse trains A, B along a timeline in accordance with the stimulation pulse train arbitration method of the invention.

FIG. 5 illustrates an exemplary stimulation pulse train that might be delivered by the IPG 104 of FIG. 1, with the stimulation pulse train having a series of monophasic rectangular stimulation pulses 300 interrupted by occasional passive balancing phases 301.

FIG. 6 illustrates an exemplary “customized” stimulation waveform that might be delivered by the IPG 104 of FIG. 1, having an approximated sinusoidal followed by a spike stimulation phase 600, followed by either a symmetrical opposite balancing phase 601 or a passive balancing phase 301 having an interphase delay 304.

FIG. 7A provides a more detailed schematic block diagram of the front end (i.e., the components immediately prior to the electrodes 102) of the IPG 104 of FIG. 1.

FIG. 7B shows an exemplary circuit that might be used for each driver 700 of FIG. 7a.

FIG. 8 illustrates an exemplary biphasic stimulation pulse train having rectangular stimulation pulses 800 followed by active balancing rectangular pulses 802, showing the resulting potential at the electrode delivering the pulses, with the shaded areas arising from irreversible Faradaic charge transfer.

FIG. 9AI illustrates the IPG 104 of FIG. 1 adapted for recordation of evoked compound action potentials (ECAPs).

FIG. 9AII shows an alternative guarded cathode stimulation configuration with associated ECAP recording.

FIG. 9B illustrates an exemplary recorded evoked compound action potential (ECAP).

FIG. 10 illustrates an exemplary intrathoracic impedance signal 1000 arising from respiration, with ECAP recording preferably occurring at pauses 1003, 1004 between inspiration 1001 and expiration 1002.

### DETAILED DESCRIPTION

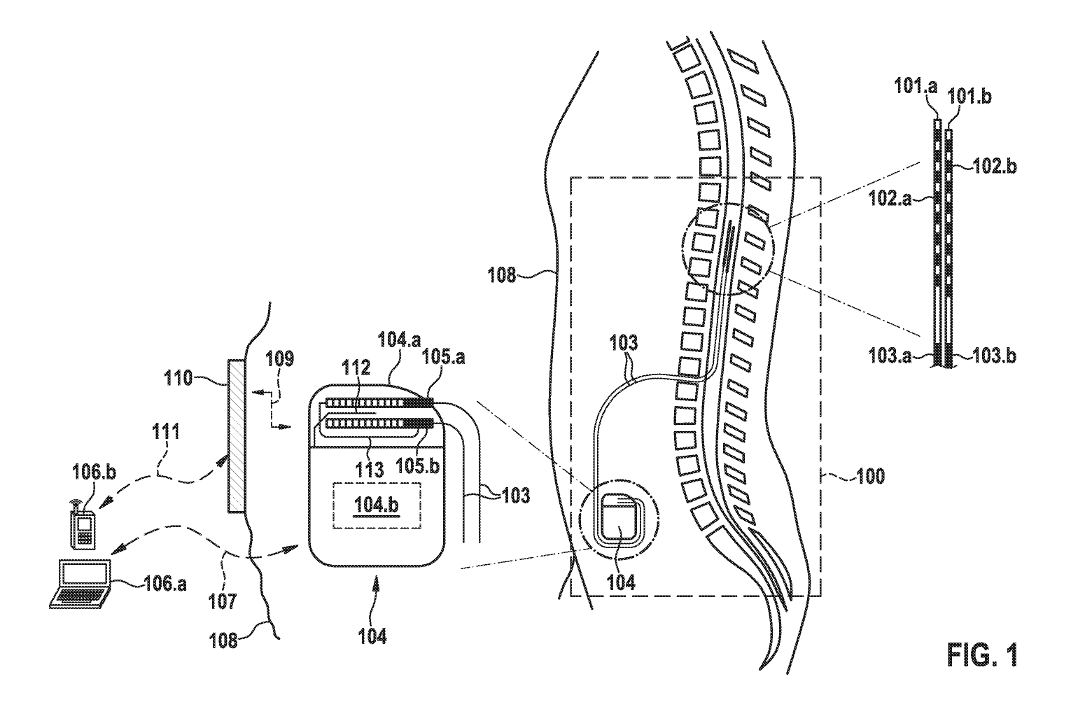

FIG. 1 illustrates an exemplary implantable spinal cord stimulation (SCS) system 100 which includes an implantable pulse generator (IPG) 104 with electrode-bearing leads 101.a and 101.b, and an external charger 110. The leads 101.a and 101.b are shown percutaneously implanted into a targeted location in a patient's epidural space, though they could be implanted elsewhere, and the leads 101 may have a configuration different from that shown (e.g., they may be replaced by paddle leads or other types of SCS leads). The distal portions of the leads 101.a and 101.b are each shown bearing octal (eight) electrodes 102.a and 102.b, though other numbers of electrodes 102 are possible. Each of these electrodes 102.a/102.b is connected to insulated wires that run inside flexible insulated carriers 103.a and 103.b. During implantation, these carriers 103.a and 103.b are tunneled to the vicinity of the IPG 104, which is typically implanted subcutaneously in the patient's lower abdominal or gluteal region. The proximal ends of the carriers 103.a and 103.b bear connectors 105.a and 105.b insertable into the header 104.a of the IPG 104 to allow conduction of electrical charge to the electrodes 102.a/102.b. The case 104.b of the IPG 104 is preferably configured such that it can approximate a reference electrode (i.e., an electrode which has a stable and well-known potential, at least over a time window of interest), as by forming it with an effective area, and of materials (e.g. fractal Ir or TiN), that make its double-layer capacitance (when implanted) much larger than that of any of the electrodes 102.a/102.b.

The IPG 104 can wirelessly communicate with external devices 106 through suitable radio frequency (such as MICS-band or Bluetooth Low Energy), inductive, or other links 107 that allow signal communication through the patient's skin 108. Exemplary external devices 106 include a clinician programmer 106.a and a patient remote 106.b.

The IPG 104 is powered by a battery, preferably one rechargeable by external means such as via transcutaneous induction from an external charger 110. The IPG's antennas 112 and 113 for wireless communication and battery recharging are preferably embedded in the IPG header 104.a, although they may be situated inside the IPG case 104.b.

The external charger 110 may be of a commonly known type, and may powered by an internal rechargeable battery to allow patient mobility while charging, and/or may be powered directly from the mains power via a power converter (e.g., when its internal battery is low). The charger 110 may itself have only a primitive user interface, and it may communicate richer/additional status information for review on a patient remote 106.b via a wired or wireless communication link 111.

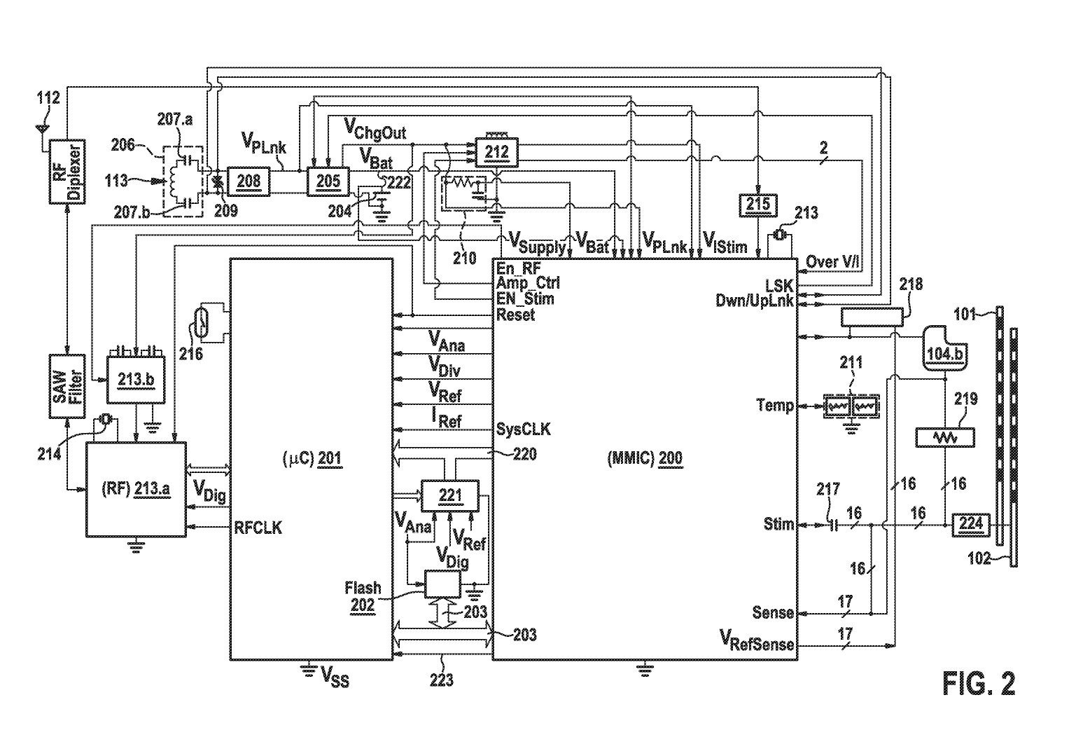

FIG. 2 shows a block diagram of exemplary architecture for the IPG electronics within the IPG case 104.b, including a mixed-mode integrated circuit (MMIC) 200, a micro-controller (pC) 201, and flash memory (Flash) 202 shared by the MMIC 200 and μC 201 (in addition to other non-volatile memory that may be included in the MMIC 200 and μC 201). A suitable interface 203, e.g. parallel I/O, permits communication between the MMIC 200 and the μC 201. The MMIC 200, μC 201, and Flash 202 are preferably packaged in a triple-stack to reduce implant size.

Additional and/or different circuits and/or arrangements may be provided depending on the capabilities of the IPG 104. As an example, the IPG 104 may have a battery charger/management circuit 205 which permits automatic “hot swapping” between voltage V_{Bat} from the battery 204 and voltage V_{PLnk} from the inductive powering receiving circuit 206 whereby the on-board electronics can be powered while the IPG battery 204 is recharged. To detect the presence of an external charger 110, the MMIC 200 may compare the voltage V_{PLnk} against a threshold (which may be programmable). When an external charger 110 is placed over the IPG 104, thereby generating a voltage V_{PLnk} higher than the programmed threshold, the MMIC 200 may inform the μC 201, and may sample V_{PLnk} to assist in optimal positioning of the charger 110 over the powering antenna (coil) 113. Once V_{PLnk} reaches the required minimum value for the battery charger/management circuit 205 to operate, the battery charger/management circuit 205 hot swaps between V_{PLnk} and V_{Bat} to power the IPG 104 (via V_{Supply}) and continue delivering therapy (if therapy is active).

The inductive powering resonant receiving circuit 206 includes an LC series resonant circuit, having antenna (coil) 113 and capacitors 207.a and 207.b, to provide current output for recharging the battery 204 upon receipt of an inductive powering link 109. A preferred operating frequency for inductive battery recharging is 130 kHz. The output of the resonant receiving circuit 206 is full-wave rectified by rectifying circuit 208 to generate V_{PLnk}, which is kept at approximately 4.9 V via feedback to the external charger 110, with a passive clamp protection 209 rated at 12.0 V. Feedback is preferably telemetered back to the external charger 110 via the inductive powering link 109 by load shift keying (LSK), for example, by changing the rectification provided by the rectifying circuit 208 from full-wave to half-wave, thus varying the reflected impedance seen by the external charger 110; by resistively/current loading V_{PLnk}; Or by untuning (capacitive modulation) the resonant circuit 206. The LSK control is implemented in the MMIC 200 hardware. Although the output voltage V_{ChgOut} of the battery charger/management circuit 205 is regulated, V_{PLnk} may be affected by the LSK communication to the external charger 110, requiring V_{ChgOut} to be further filtered (e.g., at 210) for the generation of V_{Supply} that powers MMIC 200. The filter circuit 210 also permits charge counting; the IPG 104 may include other and/or additional charge counting circuitry which is not shown in FIG. 2.

The MMIC 200 may also include one or more external thermistors 211, with two being depicted in FIG. 2, for measuring the temperatures of the IPG case 104.b and the battery 204, which can be useful when recharging the IPG battery 204. Temperature measurements may be telemetered back to the external charger 110 as part of the feedback control for the battery recharging process.

The output voltage V_{ChgOut} of the battery charger/management circuit 205 is also provided to the input of an inductive DC-DC buck-boost converter 212 that generates the required overhead voltage V_{IStim} for current-based stimulation. Preferably, V_{IStim} can vary from below the battery 204 voltage V_{Bat} and up to at least 16.0 V. To minimize power consumption, the DC-DC buck-boost converter 212 can be enabled and disabled by the MMIC 200 via output En_Stim, allowing the converter 212 to be turned on and off according to a therapy schedule. Output Amp_Ctrl, also from MMIC 200, permits adjusting the voltage V_{IStim} to the minimum required overhead for delivering the desired therapy currents. This minimum may be automatically determined by the IPG 104 control logic based on pre-measurement of the stimulating complex impedances, the minimum voltage overhead required in the sinking and sourcing current drivers, and/or the voltage build-up on the output coupling capacitors given the charge to be injected per pulse during therapy. The MMIC 200 may also temporarily program V_{IStim} to a high voltage (e.g. 15.0 V) upon detection of an over-voltage in V_{PLnk}, or upon detection of another fault condition, to protect the battery charger/management circuit 205 and downstream circuitry. This high voltage V_{IStim} permits control of pass transistors inside the battery charger/management circuit 205 for disconnection of circuitry.

The MMIC 200 preferably supports other functions for IPG 104 operation, such as system Reset, the system clock SysCLK (which may be derived from a crystal-based time-base 213), and may generate support analog voltages V_{Ana}, V_{DIV}, reference voltage V_{Ref}, and reference current I_{Ref}, for operation of the μC 201.

The IPG 104 may also include RF communication capabilities via an RF transceiver 213.a that supports a medical implant communication service (MICS) link, a BLE link, or other suitable wireless communications link 107 with an external device 106. The RF transceiver 213.a may be powered via a DC-DC step-down block 213.b (e.g. a dividing-voltage charge pump) whose input is the output voltage V_{ChgOut} of the battery charger/management circuit 205. The μC 201 provides a supporting digital voltage V_{Dig} and low frequency clock RFCLK to the RF transceiver 213.a for operation. The RF transceiver 213.a preferably has a higher-frequency time base provided by crystal 214.

The RF transceiver 213.a is preferably typically disabled or placed in a low-power state. To start an RF communications session with an external device 106, the external device 106 may send a high-power passive wakeup signal outside the band which is picked up by the wireless communications antenna 112 and detected by block 215, which alerts MMIC 200. Upon reception of a valid wakeup signal, the MMIC 200 enables power to the RF transceiver 213.a via output signal En_RF to permit communications to take place via the RF wireless communications link 107. Alternative approaches for wake-up of the RF transceiver 213.a are to use polling, advertising, and/or to trigger a reed switch 216 or magnetic sensor. A reed switch 216 may also be used to temporarily inhibit therapy or turn off/on therapy if a magnetic signal is detected for a long period of time.

As noted previously, the wireless communication link 107 may instead or additionally be an inductive communications link, e.g., for downloading firmware or performing key exchange for pairing the IPG 104 with an external device 106. Such an inductive communications link 107 is distinct from the inductive link 109 for recharging the battery 204 via the external charger 110 and the antenna (coil) 113. For firmware download and/or key exchange, the external device 106 is preferably hooked to the external charger 110 via a wired connection 111. For downlink (DwnLnk), that is, communication from an external device 106 to the IPG 104, the external device 106 sends a wake-up energy pulse to the IPG 104 (via the external charger 110) to enable listening and drive the coil of the external charger 110 in a damped fashion with a square wave preferably below 9 kHz (for FCC Part 15 waived certification). Harmonic content is picked up by the IPG resonant receiving circuit 206 with tens of mVp amplitude. The MMIC 200 can acknowledge a downloaded message by driving the IPG resonant receiving circuit 206 from a high V_{IStim} (UpLnk) in a similar fashion (i.e., damped oscillation driven by a square wave below 9 kHz), which will be picked up by the coil of the external charger 110. If desired, a separate bobbin coil (not shown) may be included in the IPG 104 inductive communication that gets shunted by the MMIC 200 when the battery 204 is being recharged.

Looking to the front-end of the IPG 104, each electrode 102 can be driven for stimulation by the MMIC 200 via output direct current (DC) blocking capacitors 217. Although only sixteen electrodes 102 are shown in FIGS. 1 and 2, the IPG 104 architecture is modular and can be extended to a larger number of electrodes 102. The MMIC 200 can also drive the IPG case 104.b for stimulation and recording. The electrodes 102 and IPG case 104.b are also connectable to the MMIC 200 via analog switches (Sense inputs) and can be individually selected for subcutaneous electrocardiogram (sECG) and evoked compound action potential-electromyogram (ECAP-EMG) recording, as discussed below. Electrodes 102 that do not participate in ECAP recording can be connected to a voltage reference V_{RefSense} generated by the MMIC 200 to perform differential ECAP recording, as discussed below. Block 218 represents standard electromagnetic interference (EMI)/defibrillation protection circuitry of the IPG 104 front-end, whereas block 224 limits current flow induced by an external magnetic field such as that of an magnetic resonance imaging (MRI) machine. Block 219, on the other hand, represents a network of high-value resistors in star configuration, as is typically used in IPG front-ends for passive charge bleed off. As will be described below, the passive resistors of block 219 can also be utilized for determination of an appropriate balancing phase of a desired stimulation pattern. The recorded sECG and ECAP-EMG are sent by the MMIC 200 to the μC 201 via a serial interface 220, whereby the μC 201 can perform further signal processing for adaptive therapy delivery. The μC 201 may include a digital signal processing (DSP) module.

The IPG 104 module may also include a triaxial accelerometer 221 which is controlled by the μC 201. The accelerometer 221 is configured to output a posture signal indicative of the patient's posture. It also allows detection of postural transitions with high sensitivity and specificity, in particular the sit-to-stand-to-sit and sit-to-lie-to-sit transitions, for automatic therapy adjustment as discussed below. Since physiological recording and posture sensing may not need to occur simultaneously, accelerometer posture sensing data may be sent to the μC 201 via the serial interface 220. The accelerometer 221 data may also be utilized for quality-of-life statistics.

The μC 201 may pre-store multiple independent therapy programs, preferably a minimum of six. Programs are typically associated with body postures (e.g. supine, on right, on left, prone, upright, and mobile), and only one is active when the IPG 104 is to provide therapy. Each therapy program may have more than one active area, preferably up to four. Switching between programs can be triggered by the patient via the patient remote 106.b prior to or following a posture change, or it can be done automatically by the IPG 104 logic based on posture information from the accelerometer 221. During therapy delivery, the μC 201 downloads the appropriate program into the MMIC 200 registers, and the MMIC 200 handles the different stimulation trains as instructed by the μC 201. In other words, all low-level timers associated with the stimulation pulses (and associated balance phases for safe electrode and tissue operation) are preferably handled by the MMIC 200, whereas all therapy management timers are preferably handled by the μC 201.

The IPG 104 architecture shown in FIG. 2 includes protective features. A fuse 222 in series with the battery 204 protects it from overcurrent conditions, and may be used for charge counting purposes. The inductive DC-DC buck-boost converter 212 has built-in over-voltage/over-current monitoring and protections for V_{IStim} and total therapy current. Detection of a faulty condition is reported to the MMIC 200 via the input Over V/I. Alternatively, monitoring circuitry may be embedded in the MMIC 200, with conditions being reported to the μC 201 via the interrupt line 223.

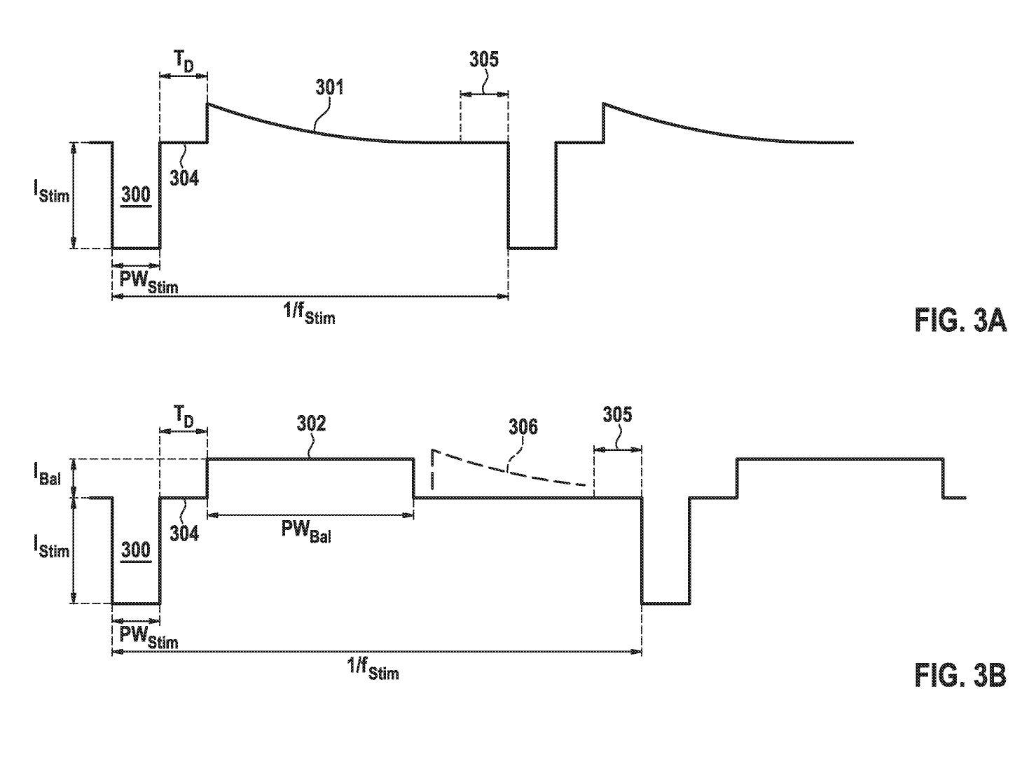

The IPG 104 can deliver stimulation phases 300, typically conventional rectangular stimulation pulses, with passive balancing phases 301 or active balancing phases 302, with examples being shown in FIG. 3a (showing passive balancing) and 3b (showing active balancing). Stimulation can be delivered via electrodes 102 and the IPG case 104.b. Each stimulation phase 300 has programmable stimulation pulse current I_{Stim} and stimulation pulse width PW_{Stim}, wherein PW_{Stim} is preferably set at a predefined time interval for all stimulation pulses in a pulse train. Their product is limited to a maximum acceptable charge injection (e.g., 12.7 μC), and each preferably has a predetermined maximum value as well (e.g., 25.0 mA and 1,000 μs). The interphase delay 304, which will be referred to as T_{D}, is also preferably programmable in the 10 μs-100 μs range. For stimulation with passive balancing phases 301, the stimulation pulse frequency f_{Stim} may preferably be programmable in the 2 Hz-220 Hz range without limitation in the stimulation pulse width PW_{Stim}, and up to 250 Hz with limits set on the stimulation pulse width PW_{Stim}. Each passive balancing phase 301 may terminate at a time 305 before the beginning of a new stimulation phase 300. This post-balancing interval 305 may be utilized to establish the programmable stimulation pulse current I_{Stim} through an internal dummy load before delivering it to tissue to avoid connection spikes in the stimulation phase 300. The post-balancing interval 305 is preferably shorter than approximately 100 μs.

The active balancing phases 302, on the other hand, are programmable via parameter balancing pulse width PW_{Bal}. The balancing pulse width PW_{Bal} is preferably set to a predefined time interval for all active balancing phases in a pulse train, and is preferably programmable as some multiple (e.g., 1×, 2×, 4×, or 8×) of the stimulation pulse width PW_{Stim}. Preferably, the balancing current I_{Bal} is determined using a determination stage as described in U.S. Provisional Patent Application 62/306,093 (the entire contents of which are incorporated by reference into this document). However, alternative methods for determining the balancing current I_{Bal} are possible, for example, it may be calculated to match the stimulation charge given by the product of the stimulation pulse current I_{Stim} and the stimulation pulse width PW_{Stim}. An auxiliary passive balancing phase 306 is added at the end of an active balancing phase 302 to further discharge tissue and the DC blocking capacitors 217.

As discussed above, the IPG 104 can automatically switch between the different stimulation programs based on the output of the triaxial accelerometer 221. Only one program may be active at any time when the IPG 104 is to deliver therapy, and each program may simultaneously stimulate with different independent trains. These trains may require sharing electrodes and different stimulation frequencies as they target different body areas. Thus, the following train arbitration method has been developed to manage potential overlapping of phases from different trains. This arbitration method permits running a pair of asynchronous trains for stimulation pulse frequencies f_{Stim} up to 130 Hz, and two pairs of trains for frequencies up to 65 Hz. The primary assumptions used for the arbitration method are:

(1) The stimulation pulse frequency f_{Stim} for each train is independently programmable.
(2) The stimulation pulse width PW_{Stim} for each train is independently programmable.
(3) The interphase delay T_{D} 304, and the post-balancing interval 305, are common in all trains.
(4) A passive balancing phase 301 must be longer than some minimum duration (e.g. 3.3 ms) to be permitted. If this minimum cannot be met by the scheduler, an active balancing phase 302 is to be delivered instead with the same stimulation pulse width PW_{Stim} as the corresponding stimulation phase 300, or with some multiple (e.g., 2×) of the stimulation pulse width PW_{Stim}.
(5) Frequency jitter in the f_{Stim(s)} during train arbitration is limited to ±20% of the corresponding programmed stimulation frequencies f_{Stim(s)}.

Looking to a basic application of the arbitration method, consider two trains A and B to be applied to different areas of the patient, with the trains running at frequencies f_{Stim} up to 130 Hz and respectively containing biphasic stimulation pulses A(n) and B(n). The stimulation pulses A(n) and B(n) can occur in any order, i.e., A(n) may be before B(n) or vice versa. During the first biphasic pulse of a set (n), the balancing phase of the second biphasic pulse of set (n) is determined. During the second biphasic pulse of a set (n), the balancing phase of the first biphasic pulse of the set (n+1) is determined instead. In both cases, periods are adjusted accordingly (i.e., frequency is jittered) to be able to deliver passive balancing 301 when possible, and switch to active balancing 302 otherwise, thereby minimizing power consumption. Balancing phases are not interrupted to deliver an upcoming stimulation phase. In the foregoing arrangement, if one train's frequency is higher than the other such that multiple pulses of one train are delivered between pulses of the other, the final one of the multiple pulses of the one train will be used to determine the balancing phase of the subsequent pulse of the other train. For example, if train B has a higher frequency than train A, if B(n) is a first biphasic pulse of a set (n) and the next biphasic pulse B(n+1) is scheduled before A(n), biphasic pulse B(n+1) is delivered and takes on the role of B(n), redefining the set (n) to resume the arbitration method.

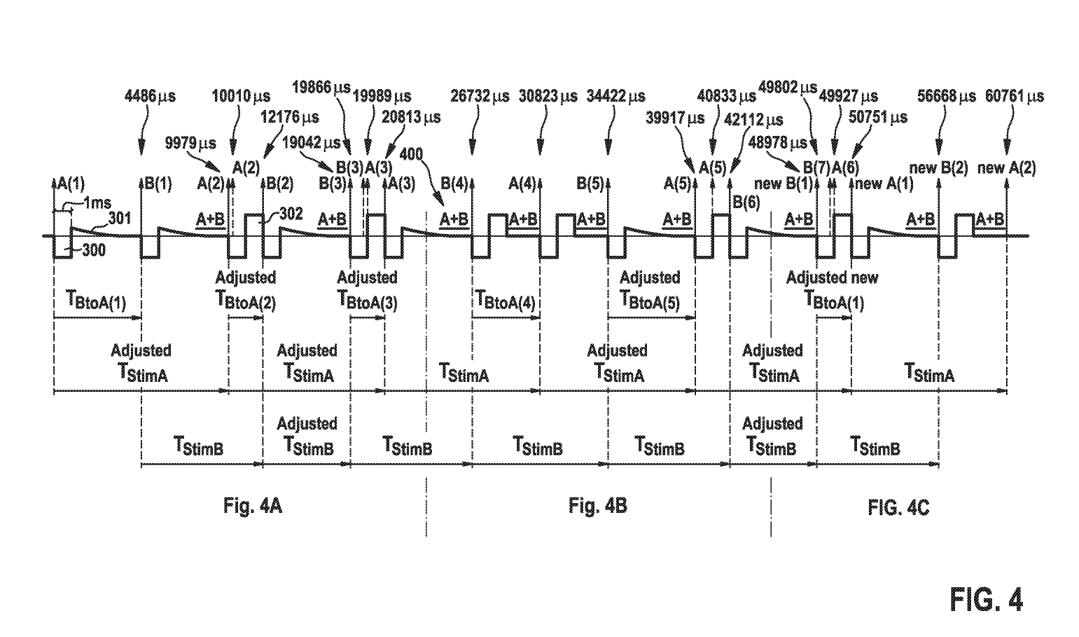

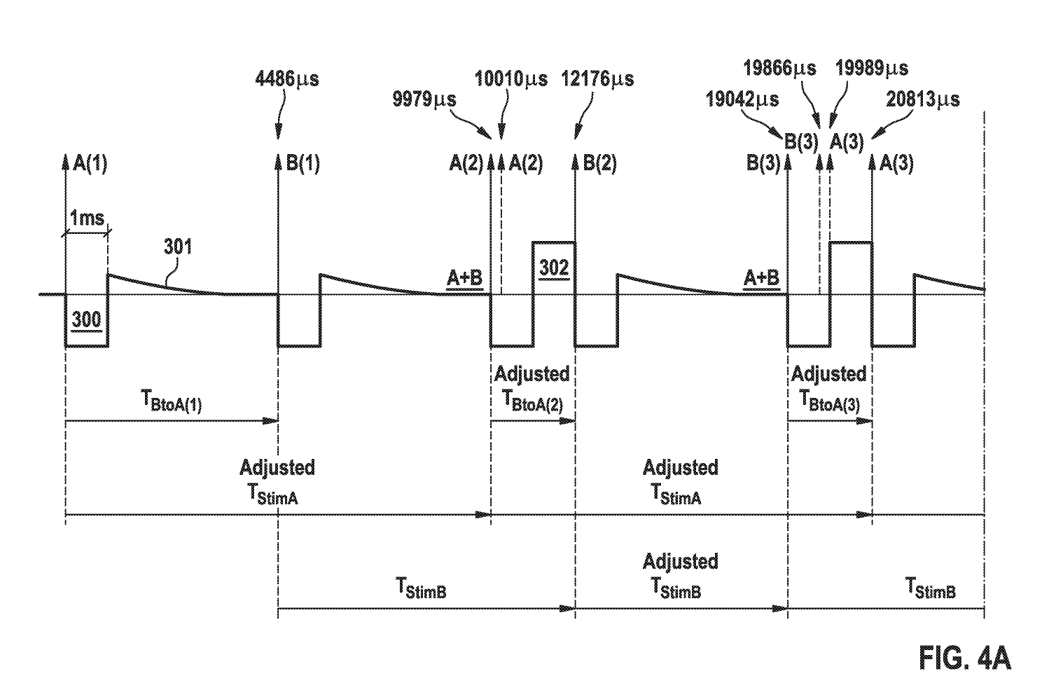

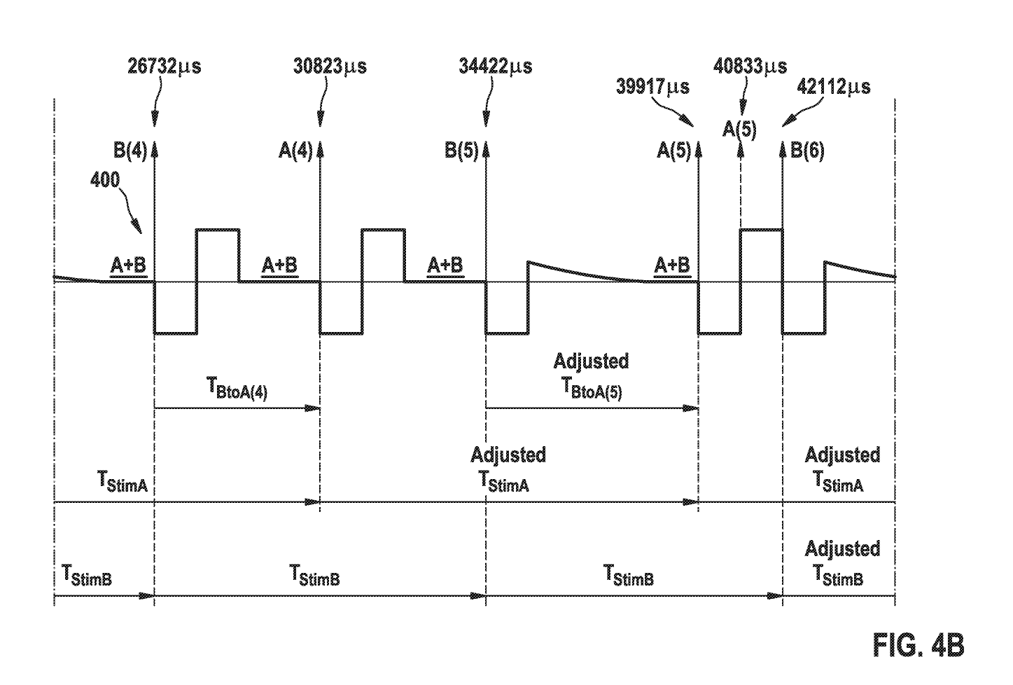

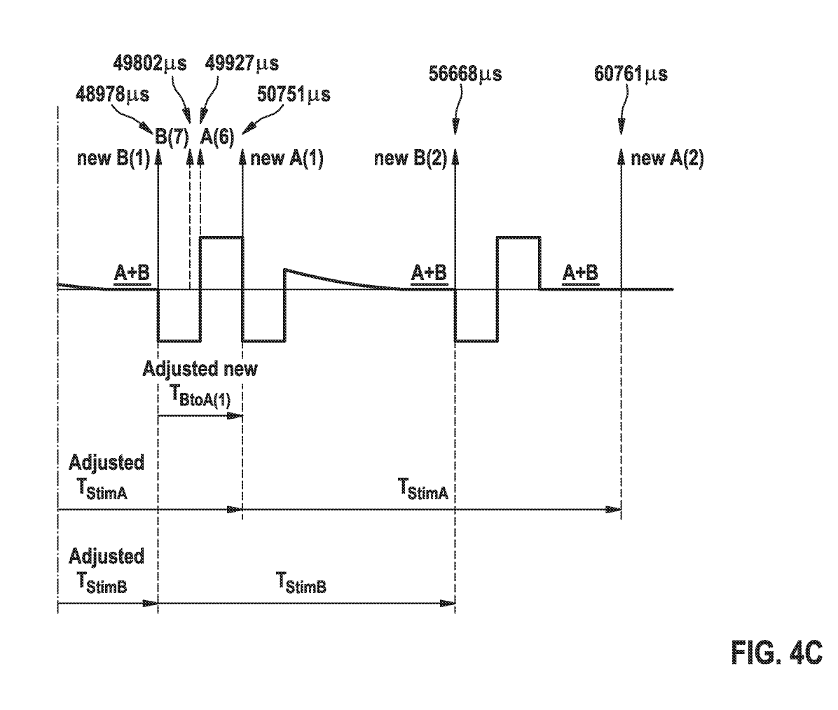

FIGS. 4-4c then show an example of the arbitration method using the following parameters, assuming the time-base (Ck32k) is nominally 32,768 Hz:

(1) Area A train nominally runs at 100 Hz (period T_{StimAnom}, or simply T_{StimA}, is 10 ms≅328 Ck32k).
(2) Area B train nominally runs at 130 Hz (period T_{StimBnom}, or simply T_{StimB}, is 7.69 ms≅252 Ck32k).
(3) Pulse widths are 1,000 μs (1 ms) each.
(4) The interphase delay T_{D} and the post-balancing interval 305 are 100 μs (0.1 ms≅3 Ck32k) each.
(5) The minimum passive balancing phase 301=3.3 ms≅108 Ck32k.

Solid arrows in FIGS. 4-4c represent the actual A(n) and B(n) delivered events, whereas dotted arrows represent where the events were scheduled to be delivered according to the programmed train parameters. Approximate times for each event are indicated in μs. The A(1) phase 300 is delivered first, followed by the B(1) pulse, both with passive balancing phases 301 (as permitted by programmed parameters). At the end of the B(1) pulse, and before the delivery of the A(2) phase, there is time indicated as A+B for simultaneously applying an auxiliary passive balancing phase 306 to both trains A and B (as timing allows it). As shown, the B(2) pulse could be delivered as scheduled, but the A(2) pulse needed to be delivered slightly earlier, and with an active balancing phase 302 instead of passive balancing 301 due to the value of T_{BtoA(2)}, wherein T_{BtoA(i)}=Time_scheduled_event B(i)−Time_scheduled_event A(i) from event B(i−1). Adjusted timers (where applicable) are indicated below the time axis by arrows between events. In the case of the A(3) and B(3) pulses, both events needed to be moved in time as shown, with B(3) occurring before A(3) (with both the original and adjusted T_{BtoA(3)} values being negative). Subsequent A(n) and B(n) pulse times can be determined in a similar manner. Other times A+B for simultaneous auxiliary passive balancing 306 of both trains A, B where possible, are also shown.

Pulse B(7) is scheduled to be delivered between B(6) and A(6). This implies delivery of pulse B(6), followed by a new B(1) pulse instead of B(7). Pulse A(6) then becomes the new A(1) pulse, and the arbitration method continues with a new set (1), and new B(1) and A(1) pulses.

The foregoing arbitration method can readily be extended to additional pairs of trains. For example, a second pair of trains C and D could be delivered parallel to the trains A and B above, with trains C and D being delivered according to the same rules governing the delivery of trains A and B.

The IPG 104 (FIG. 1) may also (or alternatively) apply other arbitration methods for the trains A and B, or for other trains. As an example, the IPG 104 might also deliver two independent pulse trains with symmetric active balance phases, with one train having a stimulation frequency f_{Stim} preferably higher than 1,300 Hz, and the second train having a stimulation frequency f_{Stim} preferably lower than 130 Hz, with the trains being interleaved (i.e., the higher-frequency train is delivered during the quiescent time of the lower-frequency train). The IPG 104 may also deliver independent trains with stimulation frequencies f_{Stim} higher than 130 Hz in alternating time periods, each one running for a programmable time, preferably in the range of 100 ms to 500 ms. For example, if there are three trains A, B, and C, train A could first be delivered for the programmed time, followed by delivery of train B for the programmed time, followed by delivery of train C for the programmed time, then restarting with train A.

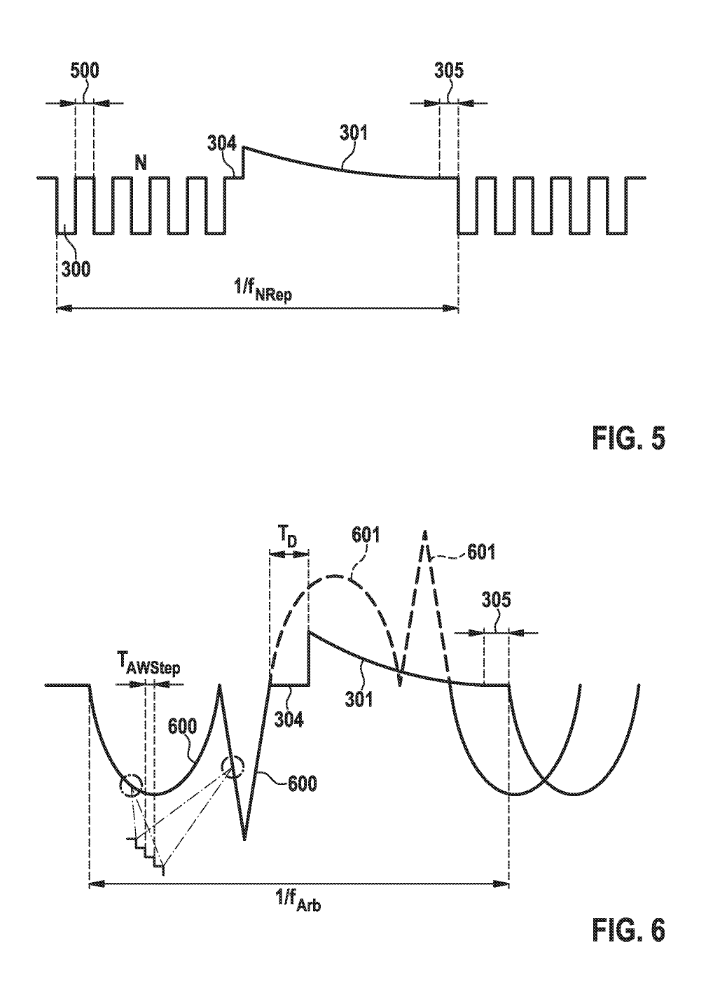

As another example, the IPG 104 could also deliver a group of N monophasic stimulation pulses 300 as shown in FIG. 5, repeated at a frequency fNRep, with a passive balancing phase 301 between consecutive groups. This pulse scheme may calm pain with reduced paresthesia. The pauses 500 between consecutive pulses 300 within a group is a programmable parameter, each being up to a few ms. The number of pulses N is preferably programmable between 2-8, and fNRep is preferably programmable between 2 Hz-60 Hz. The cumulative charge during the N pulses of a group is limited to the maximum acceptable charge injection mentioned before.

As another example, the IPG 104 could stimulate using arbitrary waveforms as shown in FIG. 6. The arbitrary waveform could be defined by a programmable (preferably graphically programmable) multi-point (e.g., 32-point) stimulation phase 600, here defining an approximated sinusoidal followed by a spike stimulation phase (with fewer or greater pulses having different shapes being possible). The stimulation phase 600 is then followed by either a symmetrical opposite balancing phase 601 or a passive balancing phase 301 having an interphase delay 304. The stimulation amplitudes, and the horizontal periods T_{AWStep} between points, are programmable parameters. T_{AWStep} is preferably programmable between 20 μs-200 μs. The frequency f_{Arb} is dictated by T_{AWStep}, the interphase delay 304, the minimum permissible passive balancing phase 301, and the post-balancing interval 305. The total charge injected during the stimulation phase 600 is again limited by the maximum acceptable charge injection.

All types of stimulation waveforms can have programmable envelope modulation, allowing the stimulation phase 300 amplitude to be ramped up and down. This feature may avoid unpleasant sensations, particularly when a train of stimulation phases 300 is first started. In addition, the IPG 104 can deliver premodulated interferential currents as described in U.S. Provisional Patent Application 62/306,094 (the entire contents of which are incorporated by reference into this document).

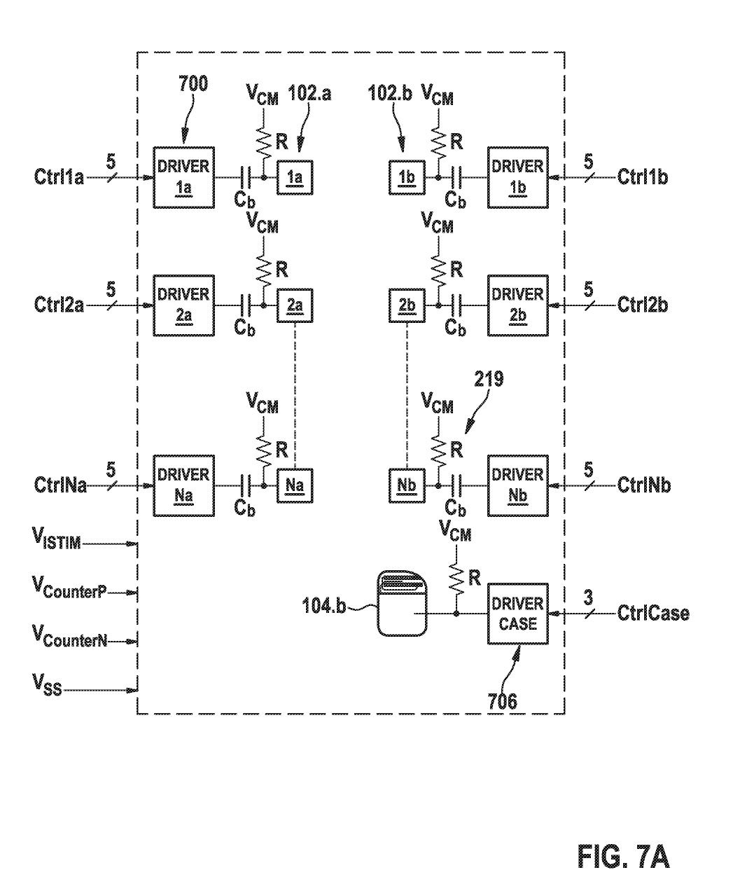

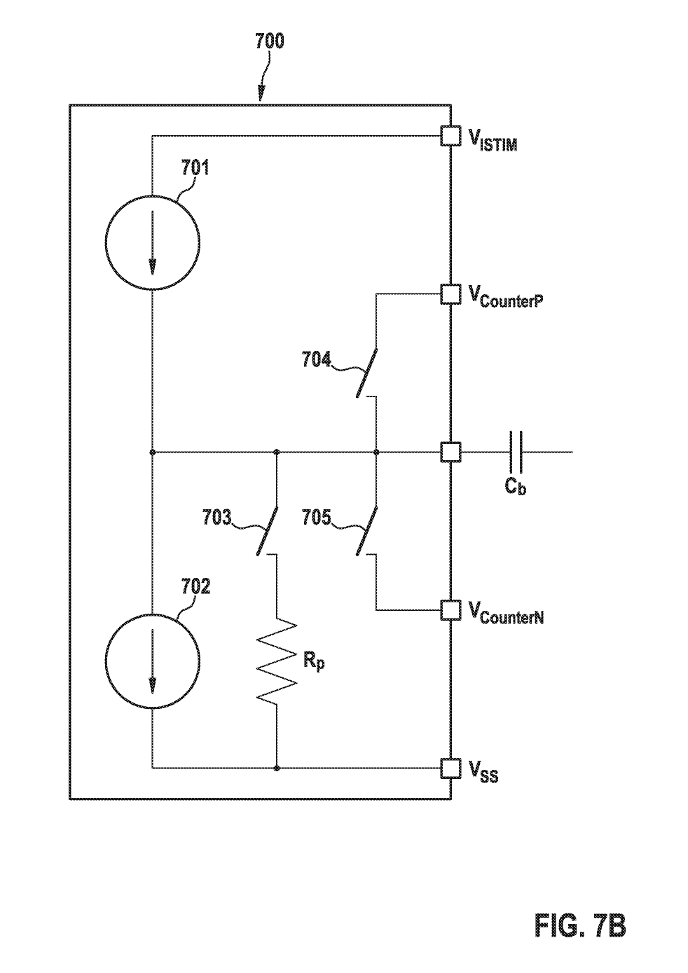

FIG. 7a presents a more detailed block diagram of the stimulation front-end of the IPG 104. Electrodes 102.a and 102.b (shown earlier in FIG. 1) are respectively represented by elements Xa and Xb (X=1 . . . N). Output DC blocking capacitors Cb are in series with each electrode Xa/Xb, and the electrodes can be driven by circuitry in drivers 700. Resistors R, connected to a common point V_{CM}, represent the individual elements of block 219 in FIG. 3. Each driver 700 has five controllable elements as shown in FIG. 7b, where only one may be active at any time when the respective electrode 102 is utilized for therapy delivery. Current 701 permits sourcing current through an electrode 102 from the programmable voltage V_{IStim}, whereas current 702 permits sinking current to V_{SS} (system ground, see FIG. 2) as desired. Having sourcing and sinking currents independently controllable at each electrode 102 permits delivery of simultaneous multi-electrode SCS therapy with active charge balancing, thereby allowing higher frequency, and also allows current steering to enable targeted stimulation of specific nerve fiber populations. For low frequency applications, analog switch 703 and current limiting resistor Rp permit a passive charge balancing phase. Although the current limiting resistor Rp is shown connected to V_{SS}, other intermediate common potentials may be utilized. Resistor Rp may be even by-passed (by an analog switch not shown) for delivering passive balance phases.

For active charge balancing, analog switches 704 and 705 permit currents to circulate from voltage V_{CounterP} or to voltage V_{CounterN} respectively. Typically, V_{CounterP} will be close to V_{IStim}, while V_{CounterN} will be close to V_{SS}, and these auxiliary voltages are generated by the MMIC 200 (FIG. 2). In some cases, depending on the impedance and programmed stimulation current, V_{CounterN} and V_{CounterP} need to be offset up to 2.0 V from V_{IStim} or V_{SS} to prevent the circuitry in drivers 700 from exceeding V_{IStim} or from going below V_{SS}, which would trigger undesired parasitic conduction of solid-state elements in the drivers 700.

The driver 706 for the IPG case 104.b, on the other hand, can merely include the analog switches 703, 704, 705 and the current limiting resistor Rp.

The control logic for the IPG 104 can deliver closed-loop stimulation that maintains safe voltages at each active therapy electrode 102, and avoids voltage runaway in the corresponding output DC blocking capacitors Cb associated with the electrodes 102. It can employ different types of closed-loop control depending on whether or not it is to deliver adaptive therapy based on evoked compound action potentials (ECAPs).

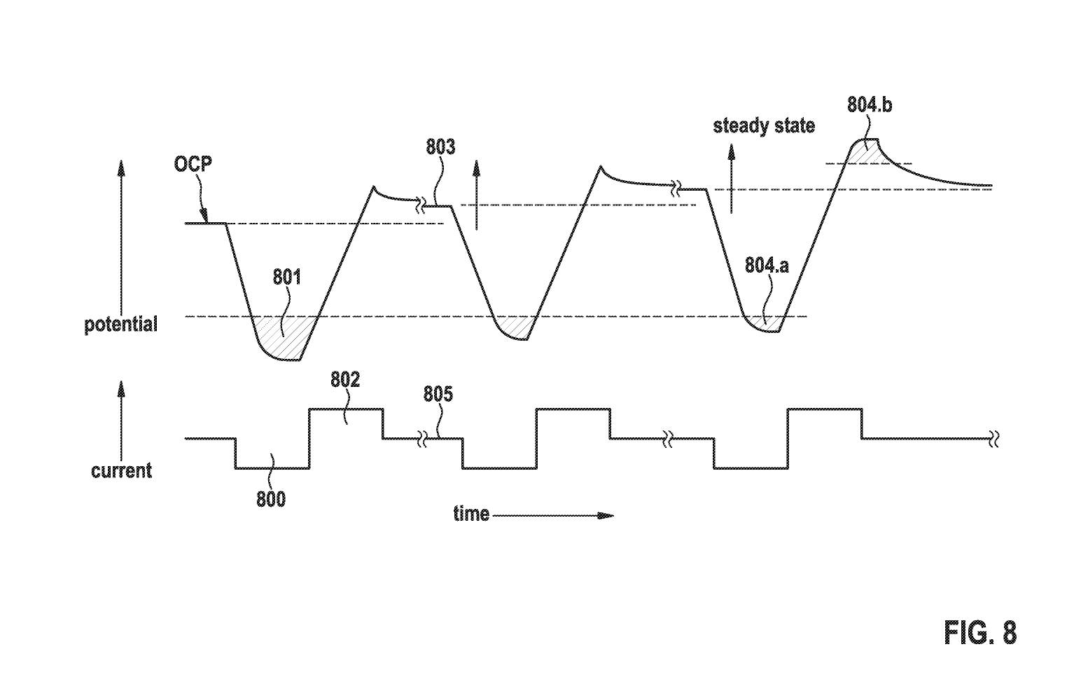

To better understand the need for closed-loop control of stimulation in applications that require multi-electrode high pulsing rate therapy (i.e., above 250 Hz or so), and where passive charge balancing 301 is not possible, refer to FIG. 8. This drawing shows the potential of a stimulating electrode 102 when active charge balancing is used. The electrode potential begins from its open circuit potential (OCP) (measured against a suitable voltage reference electrode). During delivery of the first cathodic pulse 800, the electrode-tissue double layer reversibly charges and the electrode 102 may begin to transfer charge into Faradaic reactions 801 as its potential moves negatively. Since it is likely some irreversible charge transfer will occur during the stimulation pulse 800, not all of the injected charge may go into charging the double layer under such situation. Hence, only a fraction of the cathodic charge of pulse 800 would be required during the anodic balancing phase 802 to bring the potential back to OCP. If the anodic phase 802 is charge-balanced with the cathodic phase 800 instead, as traditionally implemented in IPGs, the pre-pulse potential 803 of successive pulses moves positively until the same amount of charge is lost during the cathodic and anodic phases, at shaded areas 804.a and 804.b. If this occurs, the anodic Faradaic reaction 804.b may cause electrode corrosion. In the case of a platinum (Pt) electrode, for example, platinum oxide (PtO) may be formed, and soluble Pt compounds—including toxic products such as cisplatin [PtCl2(NH3)2]— may be generated when such PtO reacts in the chloride medium. Unbalanced charge stimulation could be used instead, but this creates voltage runaway in the output DC blocking capacitors Cb. The closed-loop stimulation of the arrangement described herein overcomes such limitations by providing a multi-electrode, multi-current system and method that delivers stimulation with a scheme that automatically adjusts the injected charges to maintain safe operation while preventing voltage runaway in the output DC blocking capacitors.

As discussed in the aforementioned U.S. Provisional Patent Application 62/306,093, closed-loop stimulation control can deliver the minimum charge imbalance needed to guarantee that at each active electrode, both its associated output DC blocking capacitors Cb and double layer capacitance (in series with Cb) charge in the same direction. Under the system proposed herein, the stimulating electrodes will charge in one direction whereas the return electrodes will charge in the opposite direction to allow compensating when certain voltage limits are reached.

Prior to therapy, the necessary imbalance may be determined for a given therapy program by first independently cycling through each programmed stimulating electrode to be used for therapy, and stimulating (in accordance with the therapy program) against a pseudo reference electrode instead (e.g., the IPG case 104.b). The IPG 104 control logic can cycle through all return electrodes except for one, which is forced to handle the current mismatches. During this “determination stage,” parameters that measure the final programmed “unbalance” for each active electrode 102 are saved, and the stimulating and return electrodes with the largest voltage drift, as well as the forced return electrode, are selected for indirect monitoring during therapy.

Once the determination stage is completed, therapy is delivered as programmed. During the open circuit phases 805 (FIG. 8) where no current is imposed by the IPG 104, comparators are used to indirectly compare the accumulated electrode-tissue double-layer voltages (of the electrodes selected for monitoring) against variable reference voltages internally generated in the IPG 104. The comparators allow monitoring the stimulating and return electrode voltages with the largest excursions, and the forced return electrode, between programmable limits without directly accessing the voltages of such electrodes 102. Once a comparator triggers, correction phases take place to start moving the accumulated charge in the opposite direction. These correction phases can either be performed by having a separate active phase during part of the open circuit phases 805, or by adjusting successive active balancing phases 302.

If adaptive therapy is to be delivered based on Evoked Compound Action Potentials (ECAPs), a finer determination stage can be implemented in the IPG 104 control logic (as described in U.S. patent application Ser. No. 15/451,838], the entire contents of which are incorporated by reference into this document). Electrical stimulation depolarizes fibers, generating propagating action potentials. ECAPs are the sum of electroneurographic (ENG) activity recorded from a number of nerve fibers when these are stimulated above threshold. ECAPs amplitudes are typically in the tens of microvolts range, and their recording is traditionally plagued with inherent electrical and other interfering signals which are orders of magnitude larger. As an example, the stimulus artifact (SA), i.e. the non-propagating voltage transient produced as a result of electrical stimulation, is coherent with the ECAP signature and thus cannot be reduced by averaging. Its amplitude may saturate the ECAP recording front-end, and its effect may extend beyond the duration of the stimulus pulse when the ECAP signature is to be recorded. Interference by the much larger electromyographic (EMG) activity of nearby muscles, and heart activity (sECG), may also affect ECAP recording. The finer determination stage described in U.S. patent application Ser. No. 15/451,838 optimizes biphasic electrical stimulation to return the post-stimulation electrode potentials close to their open circuit potentials (OCPs) to reduce the stimulus artifact component for ECAP recording. The IPG 104 may also incorporate the systems and methods disclosed in U.S. patent application Ser. No. 15/451,838 to deal with electromyographic (EMG) activity of nearby muscles, remnant stimulus artifacts (SA), and heart activity (sECG), which may further contaminate ECAP recording.

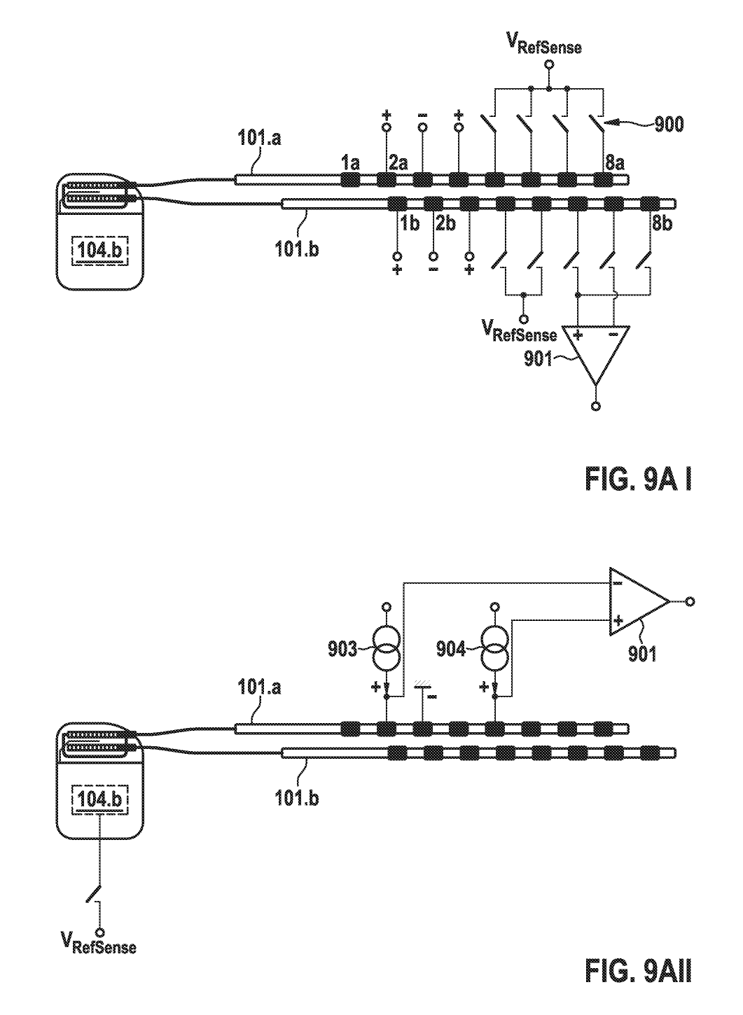

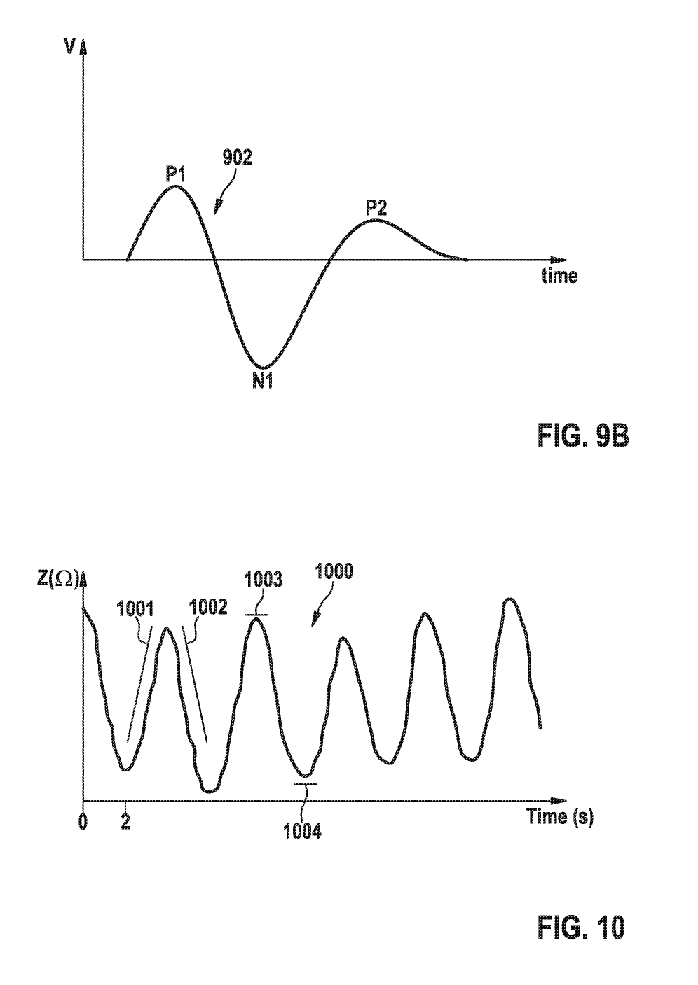

For the arrangement shown in FIG. 1, a preferred configuration for ECAP recording is the quasi-tripolar arrangement shown in FIG. 9a I This has a typical guarded cathode configuration for stimulation, i.e., 3a and 2b are stimulating electrodes which are surrounded by return electrodes 2a, 1b, 4a and 3b. Different electrodes may have different sourcing and sinking currents to steer the electrical field as desired.

Following a programmable post-stimulus blanking period, intermediate unused electrodes 5a, 6a, 7a, and 8a and 4b, 5b are connected to a voltage reference V_{RefSense} (generated by the MMIC 200) via analog switches 900, which “drives” the body common mode for recording. Blanking may be accomplished via disconnection of switches 900 and/or other methods of placing the ECAP recording front-end 901 in a state so as to minimize the artifactual effect of the blanking termination.

The distal electrodes 6b and 8b are tied together and connected to the non-inverting input of the ECAP recording front-end 901, whereas the distal center electrode 7b is connected to the inverting input instead. Preferably, the ECAP recording electrodes are selected as far away as possible from the stimulation electrodes to minimize the stimulus artifact (SA). Alternatively, recording can occur using 6a tied to 8a as one electrode, and using 7a as the other electrode, and having 5a and 4b, 5b, 6b, 7b, 8b connected to V_{RefSense} instead. The recording front-end 901 preferably presents programmable input ranges and band-pass characteristics, adjustable gain, high input impedance, low equivalent input noise level and power consumption, adequate settling time, high power supply rejection ratio (PSRR), and high common mode rejection ratio (CMRR), among other features. The recorded ECAP 902 (FIG. 9b) has a triphasic shape (P1, N1, P2 peaks) since the quasi-tripolar configuration resolves the second derivative of the evoked neural response with respect to time.

U.S. patent application Ser. No. 15/451,838 describes possible recording configurations that can be used instead of, or in addition to, those described above. Switching to a bipolar recording configuration from a quasi-tripolar (or tripolar) configuration—a possibility noted in the prior Application—permits observation of non-propagating late-response (EMG) post-ECAP signatures. This late response may allow identifying whether unwanted activation of the nociceptive reflex arc, or muscle afferents in the dorsal roots, is caused by the programmed therapy. The prior Application further teaches systems and methods for ECAP signal sampling, storage, and processing, including detection of relative lead 101 migration based on ECAPs latency changes.

FIG. 9a II shows an alternative guarded cathode stimulation configuration with associated ECAP recording as described in U.S. Patent Application 62/537,003. The desired stimulation current at the cathode (−) is injected via two current sources 903, 904a at the anodes (+). The values of these currents are adjusted, without impacting therapy, to make the voltages at the inputs of the sensing front-end 901 follow similar voltage transitories during stimulation and active balancing phases 300, 302 so they can be rejected by the high common mode rejection ratio (CMRR) of the sensing front-end 901. The asymmetry in the positioning of the anodes (+) with respect to the cathode (−) permit recording an ECAP signal. The IPG case 104.b, or other unused electrodes 102 as shown in FIG. 9a I, may be connected to voltage reference V_{RefSense} to drive the body common mode for recording. The configuration of FIG. 9a II permits recording an ECAP signal simultaneously with kHz stimulation and observing the response of stimulated tissue rather than a propagated ECAP signal as the same electrodes are utilized for stimulation and recording. Unlike prior art, this embodiment further permits closing the loop to adjust sub-perception therapy.

The IPG 104 control logic preferably synchronizes ECAP recording with respiration and cardiac refractory period for adaptive therapy. Respiration may cause variation in the distance between the electrodes 102 and the target fibers to be stimulated, thus requiring adaptation of therapy intensity in order to maintain pain suppression without side effects (e.g., unwanted muscle recruitment caused by intensity being too strong). A respiration signal is generated via electrical impedance measurement between any unused electrode 102 and the IPG case 104.b. Cardiac activity (sECG) may be also be recorded between the IPG case 104.b and any unused electrode 102. A train of sub-threshold biphasic pulses, asynchronous with respect to therapy pulses, may be utilized for impedance measurement. Effective therapy maintains the N1-P2 amplitude (FIG. 9b) within a desired window.

FIG. 10 shows breathing 1000 has alternating periods of inspiration 1001 and expiration 1002, with brief pauses 1003, 1004 therebetween. At pause 1003, the IPG 104 control logic performs an ECAP recording and adjusts (increases) the stimulation intensity (as by modifying the stimulation phase 300 amplitude) so the N1-P2 amplitude is within the desired window. The maximum N1-P2 amplitude is stored as I1003. At the other pause 1004, the IPG 104 control logic also performs an ECAP recording and adjusts (decreases) the stimulation intensity to keep the N1-P2 amplitude within the same target range. Such minimum amplitude is saved as I1004.

The IPG 104 control logic also extracts the duration of the inspiration 1001 and expiration 1002 periods. Utilizing these durations, and the saved I1003 and I1004 values, amplitude up/down ramps are automatically derived for delivery of stimulation phases 300 during each period 1001 and 1002 respectively. Ramps may not be linear in amplitude and have a number of steps, preferably in the range of 16-128. For example, if tonic SCS is delivered at the traditional frequency f_{Stim} of 40 Hz, and the duration of the expiration phase 1002 is 2 s, the stimulation phases 300 amplitude will vary in 80 steps between pauses 1003 and 1004.

The description set out above is merely of exemplary preferred versions of the invention, and it is contemplated that numerous modifications and additions can be made. These examples should not be construed as describing the only possible versions of the invention, and the true scope of the invention will be defined by the claims included in any later-filed utility patent application claiming priority from this provisional patent application.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. An implantable pulse generator configured to deliver first and second stimulation pulse trains wherein the first and second trains:
are delivered substantially simultaneously,
are delivered at different frequencies, and/or
are each biphasic, including a stimulation phase followed by a balancing phase.

2. The implantable pulse generator according to claim 1, wherein:
following each train's stimulation phase:
(1) the train's balancing phase is passively delivered if passive delivery can be completed prior to the other train's stimulation phase;
(2) else the train's balancing phase is actively delivered.

3. The implantable pulse generator according to claim 2, wherein:
the train's balancing phase is actively delivered with rescheduling of the other train's stimulation phase if:
i. passive delivery cannot be completed prior to the other train's stimulation phase, and
ii. active delivery cannot be completed prior to the other train's stimulation phase.

4. The implantable pulse generator according to claim 2 wherein:
the train's balancing phase is actively delivered without rescheduling the other train's stimulation phase if:
i. passive delivery cannot be completed prior to the other train's stimulation phase, and
ii. active delivery can be completed prior to the other train's stimulation phase.

5. The implantable pulse generator according to claim 1, further including an electrode through which both the first and second trains are delivered.

6. The implantable pulse generator according to claim 2, wherein rescheduling of any biphasic stimulation pulse jitters the trains' stimulation frequencies f_{Stim(s)} less than ±20%.

7. A method for generating electrical pulses for neurostimulation, the method comprising:
delivering a first stimulation pulse trains; and
delivering a second stimulation pulse trains.
wherein the first and second trains:
are delivered substantially simultaneously,
are delivered at different frequencies, and/or
are each biphasic, including a stimulation phase followed by a balancing phase.

8. The method for generating electrical pulses according to claim 7, wherein: following each train's stimulation phase:
(1) the train's balancing phase is passively delivered if passive delivery can be completed prior to the other train's stimulation phase;
(2) else the train's balancing phase is actively delivered.

9. The method for generating electrical pulses according to claim 8, wherein:
the train's balancing phase is actively delivered with rescheduling of the other train's stimulation phase if:
i. passive delivery cannot be completed prior to the other train's stimulation phase, and
ii. active delivery cannot be completed prior to the other train's stimulation phase.

10. The method for generating electrical pulses according to claim 8 wherein:
the train's balancing phase is actively delivered without rescheduling the other train's stimulation phase if:
i. passive delivery cannot be completed prior to the other train's stimulation phase, and
ii. active delivery can be completed prior to the other train's stimulation phase.

11. The method for generating electrical pulses according to claim 7, wherein both the first and second trains are delivered via an electrode.

12. The method for generating electrical pulses according to claim 8, wherein rescheduling of any biphasic stimulation pulse jitters the trains' stimulation frequencies less than ±20%.

13. A system comprising
an implantable medical device, wherein the implantable medical device comprises an implantable pulse generator according to claim 1;
at least one lead,
wherein at least one electrode for electrical stimulation is arranged along the elongated lead body and/or the distal end, and
wherein the lead proximal end is electrically connectable to the implantable medical device, and
wherein the implantable pulse generator is electrically connected to the electrode such that stimulation pulse trains can be delivered via the at least one electrode.
